(19) United States
(12) Patent Application Publication
(10) Pub. No.: US 2018/0008315 A1
(43) Pub. Date: Jan. 11, 2018

(54) TRACTION APPARATUSES AND SPINAL TRACTION SYSTEMS

(71) Applicant: Mayo Foundation for Medical Education and Research, Rochester, MN (US)

(72) Inventor: Bradford L. Currier, Rochester, MN (US)

(73) Assignee: Mayo Foundation for Medical Education and Research, Rochester, MN (US)

(21) Appl. No.: 15/545,547

(22) PCT Filed: Jan. 21, 2016
(86) PCT No.: PCT/US16/14231

§ 371 Date: Jul. 21, 2017

Related U.S. Application Data
(60) Provisional application No. 62/105,983, filed on Jan. 21, 2015.
(60) Provisional application No. 62/261,697, filed on Dec. 1, 2015.

(51) Int. Cl. A61B 17/64; A61B 17/66

(52) U.S. Cl. CPC A61B 17/6408; A61B 17/66; A61B 17/6433; A61B 17/6458

(57) ABSTRACT

Traction apparatuses can be used to facilitate spinal surgery. For example, this document provides traction apparatuses that engage with the skull of a patient, traction apparatuses that engage with the pelvis of a patient, and cervical traction systems for use in conjunction with the skull and/or pelvis traction apparatuses to facilitate correction of occipito-cervical-thoracic deformities, and to safely position the head during spinal surgery. Additionally, this document provides thoraco-lumbar-pelvic spinal deformity correction apparatuses to manipulate the position of the pelvis in relation to the spine during surgery.

### CROSS REFERENCE TO RELATED APPLICATIONS

This application claims the benefit of U.S. Provisional Ser. No. 62/105,983 filed Jan. 21, 2015 and U.S. Provisional Ser. No. 62/261,697 filed Dec. 1, 2015. This disclosure of the prior applications are considered part of (and are incorporated by reference in) the disclosure of this application.

### BACKGROUND

### 1. Technical Field

This document relates to traction apparatuses and systems. For example, this document relates to traction apparatuses that engage with the skull of a patient and cervical traction systems for use in conjunction with the skull traction apparatuses.

### 2. Background Information

In orthopedic medicine, traction refers to the set of mechanisms for straightening broken bones or relieving pressure on the spine and skeletal system.

Gardner-Wells tongs (“GW tongs”) are U-shaped tongs used for spinal traction. GW tongs have two pressure-controlled pins that are inserted at opposing sides of a patient's skull in the region of the temporal bones. The pins of the GW tongs engage with the patient's skull to permit application of a longitudinal force to the axis of the patient's spinal column.

GW tongs can be used in surgery for both anterior and posterior spine surgery. They are also used to reduce cervical fractures and dislocations, and to immobilize patients while awaiting more definitive treatment. GW tongs can also be used for positioning patients for posterior thoracic and lumbar surgery.

### SUMMARY

This document provides traction apparatuses. For example, this document provides traction apparatuses that engage with the skull of a patient, traction apparatuses that engage with the pelvis of a patient, and cervical traction systems for use in conjunction with the skull and/or pelvis traction apparatuses to facilitate correction of occipito-cervical-thoracic deformities, and to safely position the head during spinal surgery. Additionally, this document provides thoraco-lumbar-pelvic spinal deformity correction apparatuses to manipulate the position of the pelvis in relation to the spine during surgery.

In one implementation, a skull traction apparatus includes a framework, a first temporal pin, a second temporal pin, and an occiput pin. The first and second temporal pins are engaged with the framework to thereby be positioned and orientated so that the first and second temporal pins can be inserted at generally opposing sides of a patient's skull in the region of the patient's temporal bones. The occiput pin is engaged with the framework to thereby be positioned and orientated so that the occiput pin can be inserted at the occipital area of the patient's skull when the first and second temporal pins are inserted in the region of the patient's temporal bones. When each of the first and second temporal pins and the occiput pin are inserted in the patient's skull, the framework is configured for applying a longitudinal force to the patient's spinal column.

Such a skull traction apparatus may optionally include one or more of the following features. The framework may be a unitary construct. The framework may comprise two or more selectively engageable portions. The occiput pin may be engaged with an occipital frame member portion of the framework; the first and second temporal pins may be engaged with another portion of the framework; and the occipital frame member portion may be selectively engageable with the other portion of the framework. The occipital frame member portion may be selectively engageable with the other portion of the framework using a clamp mechanism. The occiput pin may be engaged with an occipital frame member portion of the framework; the first and second temporal pins may be engaged with another portion of the framework; and the occipital frame member portion may be movable in relation to the other portion of the framework. The occipital frame member portion may be pivotably movable in relation to the other portion of the framework using a hinge. The occiput pin may be engaged with an occipital frame member portion of the framework, and the occipital frame member portion may be adjustable such that the occiput pin's position is adjustable in relation to the first and second temporal pin locations.

In another implementation, a skull traction device includes an occipital frame member and an occiput pin engaged with the occipital frame member. The frame member is configured and operable for selective attachment to a U-shaped skull traction device that includes pins for engaging with generally opposing sides of a patient's skull in the region of the patient's temporal bones. The occiput pin is engaged with the occipital frame member to thereby be positioned and orientated so that the occiput pin can be engaged with the occipital area of the patient's skull when the occipital frame member is attached to the U-shaped skull traction device and when the pins of the U-shaped skull traction device are engaged with the patient's temporal bones.

Such a skull traction device may optionally include one or more of the following features. When the occiput pin and the pins of the U-shaped skull traction device are each engaged with the patient's skull, the combination of the occipital frame member and the U-shaped skull traction device may be configured for applying a longitudinal force to the patient's spinal column. The occipital frame member portion may be pivotably movable in relation to the U-shaped skull traction device using a hinge.

In another implementation, a method for applying a longitudinal force to a patient's spinal column includes: (i) engaging a U-shaped skull traction device with the patient's skull, wherein the U-shaped skull traction device includes first and second temporal pins that are engaged at generally opposing sides of the patient's skull in the region of the patient's temporal bones; (ii) engaging an occiput pin at the occipital area of the patient's skull, wherein the occiput pin is engaged with an occipital frame member that extends from the U-shaped skull traction device; and (iii) applying a longitudinal force to the patient's spinal column via the U-shaped skull traction device and the occipital frame member.

Such a method for applying a longitudinal force to a patient's spinal column may optionally include one or more of the following features. The U-shaped skull traction device may comprise a GW tongs. The occipital frame member may be pivotably movable in relation to the U-shaped skull traction device using a hinge. The occipital frame member may be adjustable such that the occiput pin's position is adjustable in relation to the first and second temporal pin locations. The U-shaped skull traction device and the occipital frame member may be a unitary construct.

In another implementation, a cervical traction system configured for applying a longitudinal force to a patient's spinal column includes a skull traction apparatus including one or more pins for engaging with the skull of the patient, an elongate traction force application member connectable to the skull traction apparatus, and an adjustable mechanism engaged with the traction force application member. The adjustable mechanism is adjustable to change a traction angle measured between the traction force application member and horizontal. While the traction force application member is in tension and the skull traction apparatus is engaged with the skull of the patient, the longitudinal force is applied to the patient's spinal column.

Such a cervical traction system may optionally include one or more of the following features. The adjustable mechanism may be manually adjustable. The adjustable mechanism may be motorized. The adjustable mechanism may include a pulley that is rotatably engaged with the traction force application member. The traction force application member may be a rope or a cable. The adjustable mechanism may be configured to be mounted in a fixed relationship with an operating table on which the patient can be placed. The system may also include one or more sensors for detecting the traction angle. The adjustable mechanism may include a motor for adjusting the traction angle. The system may also include a control system for receiving input from the one or more sensors and for sending output to operate the motor in response to the input. The control system may operate the cervical traction system to attain a selected traction angle that is input to the control system. The system may also include software for automatically correcting spinal deformities of the patient during surgery. The system may also include a pelvis manipulation apparatus for engaging with a pelvis of the patient such that the pelvis manipulation apparatus can maneuver the pelvis. The system may further comprise bracketry attachable to the skull traction apparatus and to a stationary object such that the bracketry releasably fixes the skull traction apparatus to the stationary object to hold the skull traction apparatus stationary. The stationary object may be an operating table or framework attached to the operating table.

Particular embodiments of the subject matter described in this document can be implemented to realize one or more of the following advantages. First, the skull traction apparatuses provided herein facilitate enhanced control for positioning the patient's head so that the patient's spine can be fused in the most appropriate position. The skull traction apparatuses facilitate essentially the same extent of control as a halo device, but with one fewer pin. In addition, the skull traction apparatuses provided herein facilitate easier insertion than a halo device, and cause no face scarring. The cervical traction systems provided herein can facilitate correction of occipito-cervical-thoracic deformities during spinal surgery.

Second, the cervical traction systems provided herein can safely position the head during spinal surgery. In some embodiments, the cervical traction systems provided herein are automated and can be used to change the position of the head in flexion and extension in accordance with parameters that are uploaded to the system either manually or automatically. In some embodiments, the cervical traction systems provided herein can manipulate the head and spine through all six degrees of freedom by way of an actuator or multiple actuators. In some embodiments, a software program is used in conjunction with the cervical traction systems provided herein to allow spinal deformities to be corrected automatically during the course of surgery.

Third, the thoraco-lumbar-pelvic spinal deformity correction apparatuses provided herein can be advantageously used to manipulate the position of the pelvis in relation to the spine during surgery. Accordingly, thoraco-lumbar-pelvic deformities can be corrected utilizing the devices and systems provided herein.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described herein. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description herein. Other features, objects, and advantages of the invention will be apparent from the description and drawings, and from the claims.

### DESCRIPTION OF THE DRAWINGS

FIG. 1 is a perspective view of a patient lying in a prone position and receiving spinal traction using an example skull traction device in accordance with some embodiments.

FIG. 2 is a perspective view of the skull traction device of FIG. 1.

FIG. 3 is a perspective view of another skull traction device in accordance with some embodiments.

FIG. 4 is a perspective view of another skull traction device in accordance with some embodiments.

FIG. 5 is a perspective view of another skull traction device in accordance with some embodiments.

FIG. 6A is a perspective view of another skull traction device in accordance with some embodiments.

FIG. 6B is a transverse cross-sectional view of a portion of the skull traction device of FIG. 6A.

FIG. 6C is a longitudinal cross-sectional view of a portion of the skull traction device of FIG. 6A.

FIG. 7 is a first perspective view of the skull traction device of FIG. 6A in use with an example cervical traction system in accordance with some embodiments.

FIG. 8 is a second perspective view of the skull traction device of FIG. 6A in use with the cervical traction system of FIG. 7.

FIG. 9 is a perspective view of a portion of the cervical traction system of FIG. 7 in accordance with some embodiments.

FIG. 10 is another perspective view of a portion of the cervical traction system of FIG. 7 in accordance with some embodiments.

FIG. 11 is a perspective view of the skull traction device of FIG. 6A coupled to an example interface portion for adjustably affixing the skull traction device to a stationary object.

FIG. 12 is a perspective view of the skull traction device of FIG. 6A coupled to example bracketry for adjustably affixing the skull traction device to a stationary object.

Like reference numbers represent corresponding parts throughout.

### DETAILED DESCRIPTION

This document provides traction apparatuses. For example, this document provides traction apparatuses that engage with the skull of a patient, traction apparatuses that engage with the pelvis of a patient, and cervical traction systems for use in conjunction with the skull and/or pelvis traction apparatuses to facilitate correction of occipito-cervical-thoracic deformities, and to safely position the head during spinal surgery. Additionally, this document provides thoraco-lumbar-pelvic spinal deformity correction apparatuses to manipulate the position of the pelvis in relation to the spine during surgery. In comparison to prior art skull traction devices (e.g., GW tongs), the skull traction apparatuses provided herein facilitate better control of head position during spine surgery so patients are decompressed, fused, and/or stabilized in the optimal position. In comparison to GW tongs which include two pins that are inserted at opposing sides of a patient's skull, the skull traction apparatuses provided herein include one or more additional point(s) of fixation. The additional point(s) of fixation are to the occiput area (the back of the patient's skull).

The traction apparatuses and systems provided herein are effective for use in various applications, but it is anticipated that the traction apparatuses and systems will be most commonly used for positioning patients during posterior cervical spine surgery. The cervical traction systems will manipulate the skull traction apparatuses in order to change the position of the head relative to the torso in various planes.

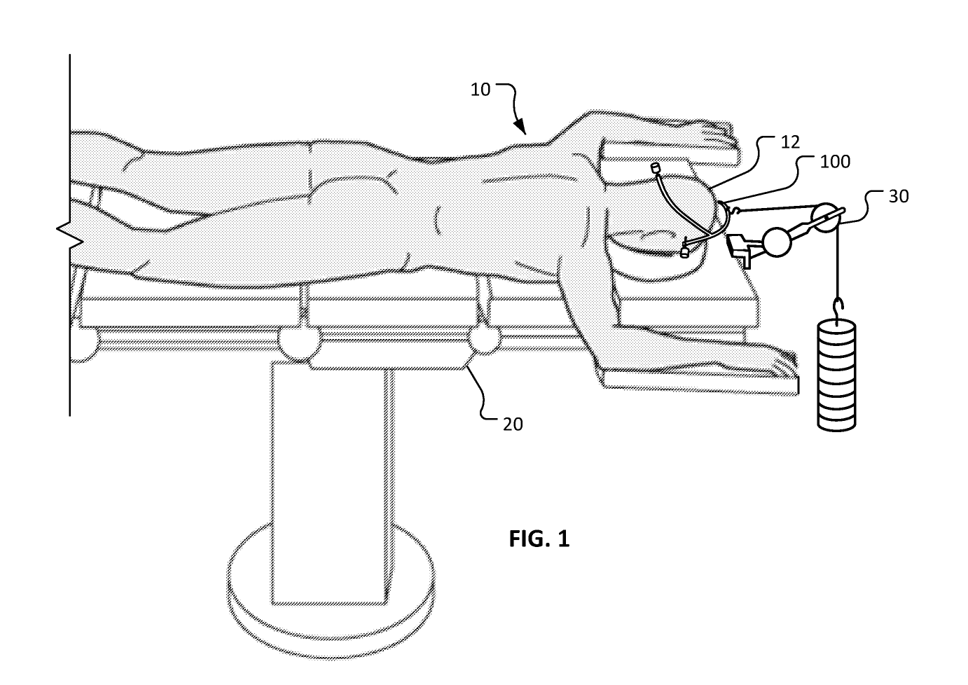

Referring to FIG. 1, a patient 10 is shown lying prone on an operating (OR) table 20. Such an orientation may be used, for example, during posterior cervical spine surgery. Engaged with patient's skull/head 12 is an example skull traction device 100. Skull traction device 100 is, in turn, coupled to a traction system 30 that is mounted to OR table 20. The combination of skull traction device 100 and traction system 30 facilitates control of patient's skull 12 during surgery (e.g., cervical surgery) so that patient 10 can be decompressed, fused, and/or stabilized in the optimal position for surgery.

The surgeon would attach skull traction device 100 after patient 10 is placed prone on OR table 20, and traction 30 would be applied to support the weight of the head 12. As described further below, skull traction device 100 facilitates three-point fixation of the skull 12, and allows the head of patient 10 to be positioned with enhanced control of flexion/extension and protrusion/retraction of the head in comparison to other devices, such as GW tongs for example. In some circumstances, skull traction device 100 is also useful for patients positioned in the supine and/or lateral positions.

In some embodiments, as described further herein, the traction system may include mechanisms for adjusting a traction angle (i.e., the angle at which the traction force is applied by the traction system in relation to horizontal).

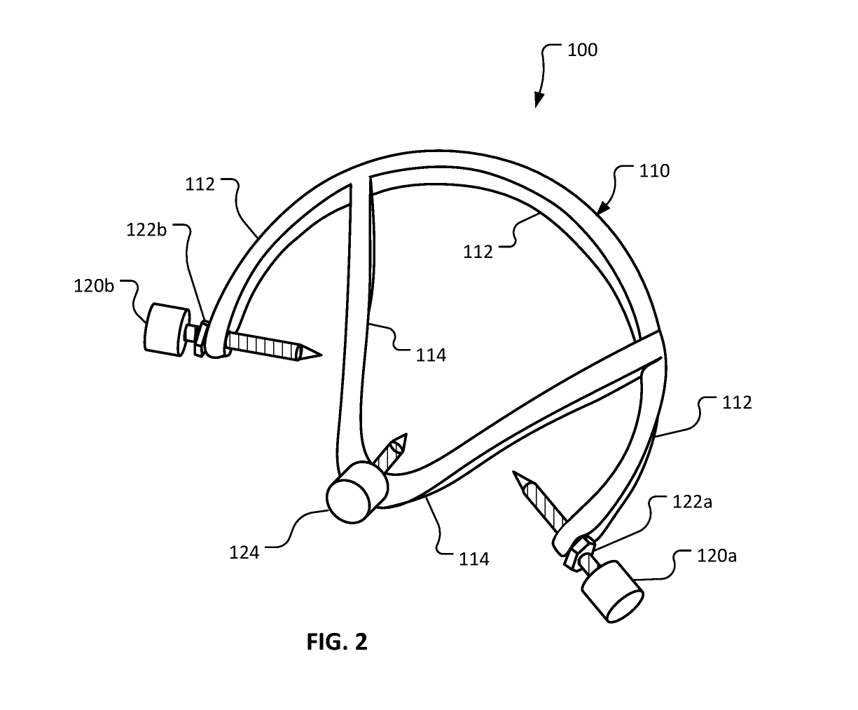

Referring to FIG. 2, skull traction device 100 provides three-point fixation. In general, skull traction device 100 includes a framework 110 and three pressure-controlled pins: a first temporal pin 120a, a second temporal pin 120b, and an occiput pin 124. First temporal pin 120a and second temporal pin 120b are generally in apposition to each other. Occiput pin 124 is disposed essentially equidistant between first temporal pin 120a and second temporal pin 120b.

In some embodiments, the pins 120a, 120b, and 124 are threaded and are engaged with complementary threaded holes in framework 110. As such, the positions of pins 120a, 120b, and 124 are adjustable in relation to framework 110. A clinician can thereby adjust pins 120a, 120b, and 124 as desired to appropriately engage skull traction device 100 with the skull of a patient. In some embodiments, pins 120a, 120b, and 124 include a locking device whereby pins 120a, 120b, and/or 124 can be releasably fixed in a position in relation to framework 110. For example, in the depicted embodiment locknuts 122a and 122b are included. A locknut may also be included in conjunction with occiput pin 124. In some embodiments, other types of pin locking devices may be included.

In the depicted embodiment, framework 110 includes a U-shaped portion 112 that spans between first temporal pin 120a and second temporal pin 120b. U-shaped portion 112, by itself, is essentially configured as a GW tongs. Added to U-shaped portion 112 is an occipital frame member 114. Occipital frame member 114 extends from U-shaped portion 112 in an orientation so that occipital frame member 114 supports and positions occiput pin 124 to engage with the occipital bone of a patient in the midline. While in the depicted embodiment a single occiput pin 124 is included, in some embodiments two or more occiput pins 124 are included. When multiple occiput pins 124 are included, the multiple pins 124 can be each located on the midline, or the multiple pins 124 can be elsewise orientated generally symmetrically in relation to the midline (e.g., two pins 124 that are laterally positioned on each side of the midline, and so on).

In some embodiments, framework 110 is made of materials such as, but not limited to, stainless steel, graphite, carbon fiber, steel alloys, polymeric materials, and the like, and combinations thereof. In some embodiments, one portion of framework 110 is made of one type of material and another portion of framework 110 is made of a different type of material.

Occipital frame member 114 can be engaged with U-shaped portion 112 either permanently or temporarily. For example, in some embodiments occipital frame member 114 is essentially permanently integral with U-shaped portion 112 such that the framework 110 is a unitary construct. In some such embodiments, occipital frame member 114 and U-shaped portion 112 can be constructed together by methods such as, but not limited to, welding, fastening, and by machining or casting framework 110 as a unitary member, and the like.

In some embodiments, occipital frame member 114 and U-shaped portion 112 are selectively engageable in relation to each other. That is, in some embodiments occipital frame member 114 and U-shaped portion 112 can be joined together, and also separated from each other, as desired. In particular embodiments, U-shaped portion 112 is a standard GW tongs and occipital frame member 114 is configured to be separable and selectively engageable therewith. In some such embodiments, occipital frame member 114 and U-shaped portion 112 are selectively engageable together using techniques such as, but not limited to, clamping, screwing, pining, locking, using collets, using interlocking components (e.g., dovetail joints), and the like.

In some embodiments that are selectively engageable, the position and orientation of occipital frame member 114 in relation to U-shaped portion 112 is adjustable. For example, in some embodiments occipital frame member 114 is hinged (pivotable) in relation to U-shaped portion 112. Such adjustability can allow the skull traction devices provided herein to advantageously accommodate different skull anatomy.

While in the depicted embodiment occipital frame member 114 is essentially V-shaped, it should be understood that the shape of occipital frame member 114 is not limited to the shape depicted. For example, in another embodiment occipital frame member 114 spans between first temporal pin 120a and second temporal pin 120b in a U-shape, and in a plane that allows occipital frame member 114 to support and position occiput pin 124 to engage with the occipital bone of a patient in the midline.

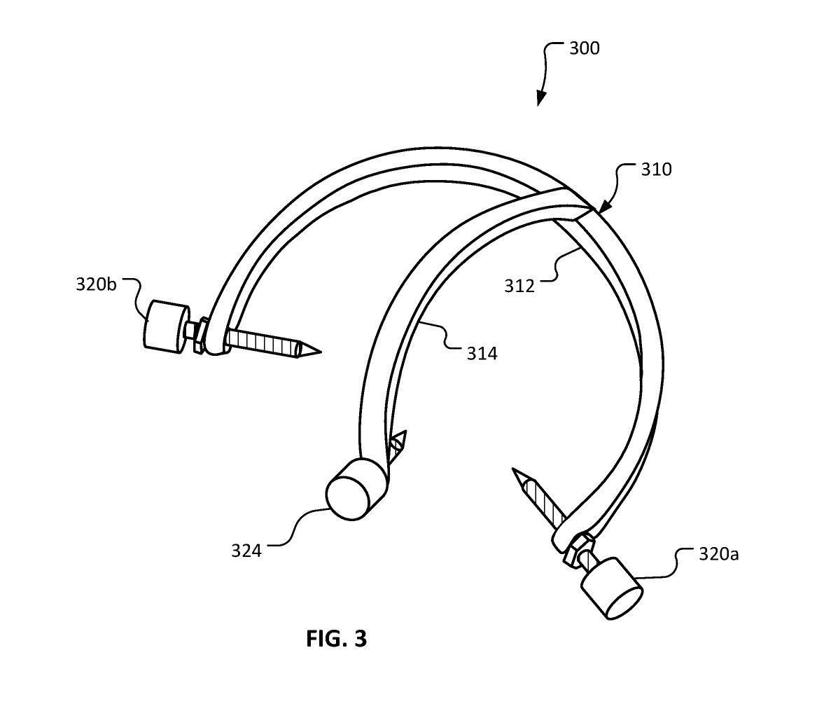

Referring to FIG. 3, another example skull traction device 300 provides three-point fixation. In general, skull traction device 300 includes a framework 310 and three pressure-controlled pins: a first temporal pin 320a, a second temporal pin 320b, and an occiput pin 324. First temporal pin 320a and second temporal pin 320b are generally in apposition to each other. Occiput pin 324 is disposed essentially equidistant between first temporal pin 320a and second temporal pin 320b.

Framework 310 includes a U-shaped portion 312 that spans between first temporal pin 320a and second temporal pin 320b. U-shaped portion 312, by itself, is essentially configured as a GW tongs. Added to U-shaped portion 312 is an occipital frame member 314. Occipital frame member 314 extends from U-shaped portion 312 in an orientation so that occipital frame member 314 supports and positions occiput pin 324 to engage with the occipital bone of a patient in the midline. While in the depicted embodiment a single occiput pin 324 is included, in some embodiments two or more occiput pins 324 are included. When multiple occiput pins 324 are included, the multiple pins 324 can be each located on the midline, or the multiple pins 324 can be elsewise orientated generally symmetrically in relation to the midline (e.g., two pins 324 that are laterally positioned on each side of the midline, and so on).

U-shaped portion 312 and first and second temporal pins 320a and 320b can be constructed and configured analogous to U-shaped portion 112 and first and second temporal pins 120a and 120b of skull traction device 100 as described above. In some embodiments, U-shaped portion 312 and first and second temporal pins 320a and 320b are a conventional GW tongs.

Occipital frame member 314 can be engaged with U-shaped portion 312 either permanently or temporarily. For example, in some embodiments occipital frame member 314 is essentially permanently integral with U-shaped portion 312 such that the framework 310 is a unitary construct. In some such embodiments, occipital frame member 314 and U-shaped portion 312 can be constructed together by methods such as, but not limited to, welding, fastening, and by machining or casting framework 310 as a unitary member, and the like.

In some embodiments, occipital frame member 314 and U-shaped portion 312 are selectively engageable in relation to each other. That is, in some embodiments occipital frame member 314 and U-shaped portion 312 can be joined together, and also separated from each other, as desired. In particular embodiments, U-shaped portion 312 is a standard GW tongs and occipital frame member 314 is configured to be separable and selectively engageable therewith. In some such embodiments, occipital frame member 314 and U-shaped portion 312 are selectively engageable together using techniques such as, but not limited to, clamping, screwing, pining, locking, using collets, using interlocking components (e.g., dovetail joints), and the like.

In some embodiments that are selectively engageable, the position and orientation of occipital frame member 314 in relation to U-shaped portion 312 is adjustable. For example, in some embodiments occipital frame member 314 is hinged (pivotable), slidable, articulable, or other adjustable in relation to U-shaped portion 312. Such adjustability can allow the skull traction devices provided herein to advantageously accommodate different skull anatomy.

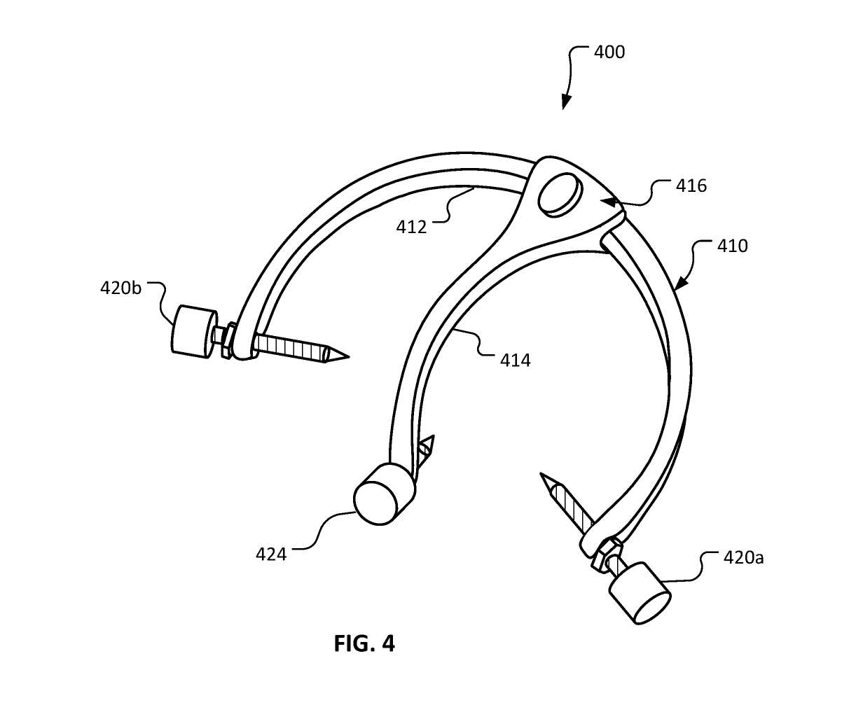

Referring to FIG. 4, another example skull traction device 400 provides three-point fixation. In general, skull traction device 400 includes a framework 410 and three pressure-controlled pins: a first temporal pin 420a, a second temporal pin 420b, and an occiput pin 424. First temporal pin 420a and second temporal pin 420b are generally in apposition to each other. Occiput pin 424 is disposed essentially equidistant between first temporal pin 420a and second temporal pin 420b.

Framework 410 includes a U-shaped portion 412 that spans between first temporal pin 420a and second temporal pin 420b. U-shaped portion 412, by itself, is essentially configured as a GW tongs. Added to U-shaped portion 412 is an occipital frame member 414 that is selectively engageable with the U-shaped portion 412 in this embodiment. Occipital frame member 414 extends from U-shaped portion 412 in an orientation so that occipital frame member 414 supports and positions occiput pin 424 to engage with the occipital bone of a patient in the midline. While in the depicted embodiment a single occiput pin 424 is included, in some embodiments two or more occiput pins 424 are included. When multiple occiput pins 424 are included, the multiple pins 424 can be each located on the midline, or the multiple pins 424 can be elsewise orientated generally symmetrically in relation to the midline (e.g., two pins 424 that are laterally positioned on each side of the midline, and so on).

U-shaped portion 412 and first and second temporal pins 420a and 420b can be constructed and configured analogous to U-shaped portion 112 and first and second temporal pins 120a and 120b of skull traction device 100 as described above. In some embodiments, U-shaped portion 412 and first and second temporal pins 420a and 420b are a conventional GW tongs and occipital frame member 414 is selectively engageable with the U-shaped portion 412.

Occipital frame member 414 can be engaged with U-shaped portion 412 either permanently or temporarily. For example, in some embodiments occipital frame member 414 is essentially permanently integral with U-shaped portion 412 such that the framework 410 is a unitary construct. In some such embodiments, occipital frame member 414 and U-shaped portion 412 can be constructed together by methods such as, but not limited to, welding, fastening, and by machining or casting framework 410 as a unitary member, and the like.

In the depicted embodiment, occipital frame member 414 and U-shaped portion 412 are selectively engageable in relation to each other. That is, in the depicted embodiment occipital frame member 414 and U-shaped portion 412 can be joined together using a joining mechanism 416, and also separated from each other, as desired. In particular embodiments, U-shaped portion 412 is a standard GW tongs and occipital frame member 414 is configured to be separable and selectively engageable therewith. In some such embodiments, occipital frame member 414 and U-shaped portion 412 are selectively engageable together by joining mechanism 416 that can be configured to use techniques such as, but not limited to, clamping, screwing, pining, locking, using collets, using interlocking components (e.g., dovetail joints), and the like.

In some embodiments that are selectively engageable, the position and orientation of occipital frame member 414 in relation to U-shaped portion 412 is adjustable. For example, in some embodiments occipital frame member 414 is hinged (pivotable), slidable, articulable, or other adjustable in relation to U-shaped portion 412. Such adjustability can allow the skull traction devices provided herein to advantageously accommodate different skull anatomy.

Referring to FIG. 5, another example skull traction device 500 provides three-point fixation. In general, skull traction device 500 includes a framework 510 and three pressure-controlled pins: a first temporal pin 520a, a second temporal pin 520b, and an occiput pin 524. First temporal pin 520a and second temporal pin 520b are generally in apposition to each other. Occiput pin 524 is disposed essentially equidistant between first temporal pin 520a and second temporal pin 520b.

Framework 510 includes a U-shaped portion 512 that spans between first temporal pin 520a and second temporal pin 520b. U-shaped portion 512, by itself, is essentially configured as a GW tongs. Added to U-shaped portion 512 is an occipital frame member 514 that is engaged with the U-shaped portion 512. Occipital frame member 514 extends from U-shaped portion 512 in an orientation so that occipital frame member 514 supports and positions occiput pin 524 to engage with the occipital bone of a patient in the midline. While in the depicted embodiment a single occiput pin 524 is included, in some embodiments two or more occiput pins 524 are included. When multiple occiput pins 524 are included, the multiple pins 524 can be each located on the midline, or the multiple pins 524 can be elsewise orientated generally symmetrically in relation to the midline (e.g., two pins 524 that are laterally positioned on each side of the midline, and so on).

U-shaped portion 512 and first and second temporal pins 520a and 520b can be constructed and configured analogous to U-shaped portion 112 and first and second temporal pins 120a and 120b of skull traction device 100 as described above. In some embodiments, U-shaped portion 512 and first and second temporal pins 520a and 520b are a conventional GW tongs and occipital frame member 514 is selectively engageable with the U-shaped portion 512.

Occipital frame member 514 can be engaged with U-shaped portion 512 either permanently or temporarily. For example, in some embodiments occipital frame member 514 is essentially permanently integral with U-shaped portion 512 such that the framework 510 is a unitary construct. In some such embodiments, occipital frame member 514 and U-shaped portion 512 can be constructed together by methods such as, but not limited to, welding, fastening, and by machining or casting framework 510 as a unitary member, and the like.

In particular embodiments, U-shaped portion 512 is a standard GW tongs and occipital frame member 514 is configured to be separable and selectively engageable therewith. In some such embodiments, occipital frame member 514 and U-shaped portion 512 are selectively engageable together by joining mechanism that can be configured to use techniques such as, but not limited to, clamping, screwing, pining, locking, using collets, using interlocking components (e.g., dovetail joints), and the like. In some embodiments that are selectively engageable, the position and orientation of occipital frame member 514 in relation to U-shaped portion 512 is adjustable. For example, in some embodiments occipital frame member 514 is hinged (pivotable), slidable, articulable, or other adjustable in relation to U-shaped portion 512. Such adjustability can allow the skull traction devices provided herein to advantageously accommodate different skull anatomy.

In the depicted embodiment, a portion of the occipital frame member 514 is adjustable. That is, in the depicted embodiment occipital frame member 514 includes an adjustable mechanism 516 and an adjustable portion 518 with which occiput pin 524 is engaged. In some embodiments, adjustable mechanism 516 is a clamp and adjustable portion 518 slides within the adjustable mechanism 518. In some embodiments, adjustable mechanism 516 includes one or more gears and adjustable portion 518 includes complementary gear teeth. The position of adjustable portion 518 in relation to adjustable mechanism 516 can be adjusted by manipulating the gears of adjustable mechanism 518. In some embodiments, adjustable mechanism 516 and adjustable portion 518 are configured to use other types of adjustable mechanisms such as, but not limited to, ratchets and pawls, lead screws, locking collets, and the like.

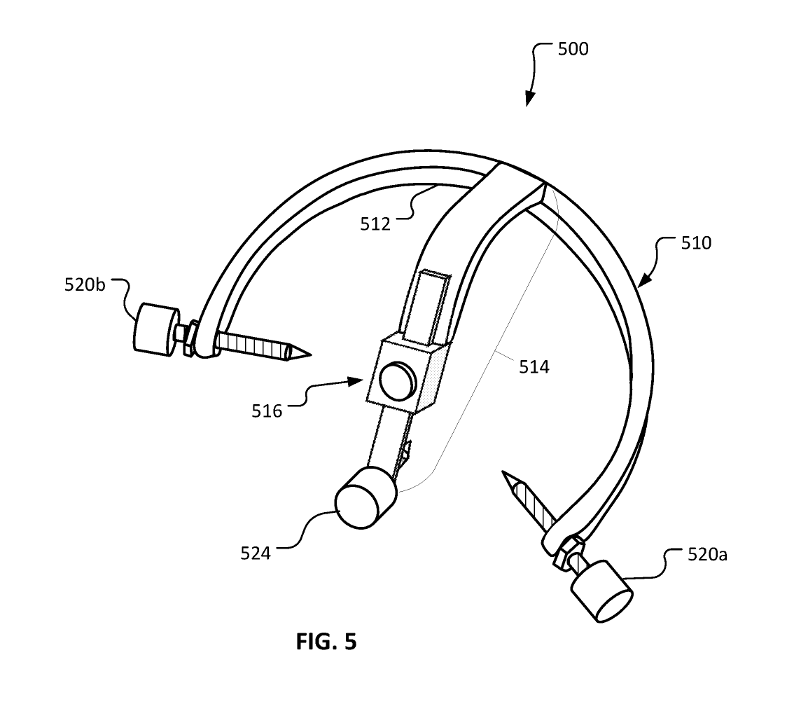

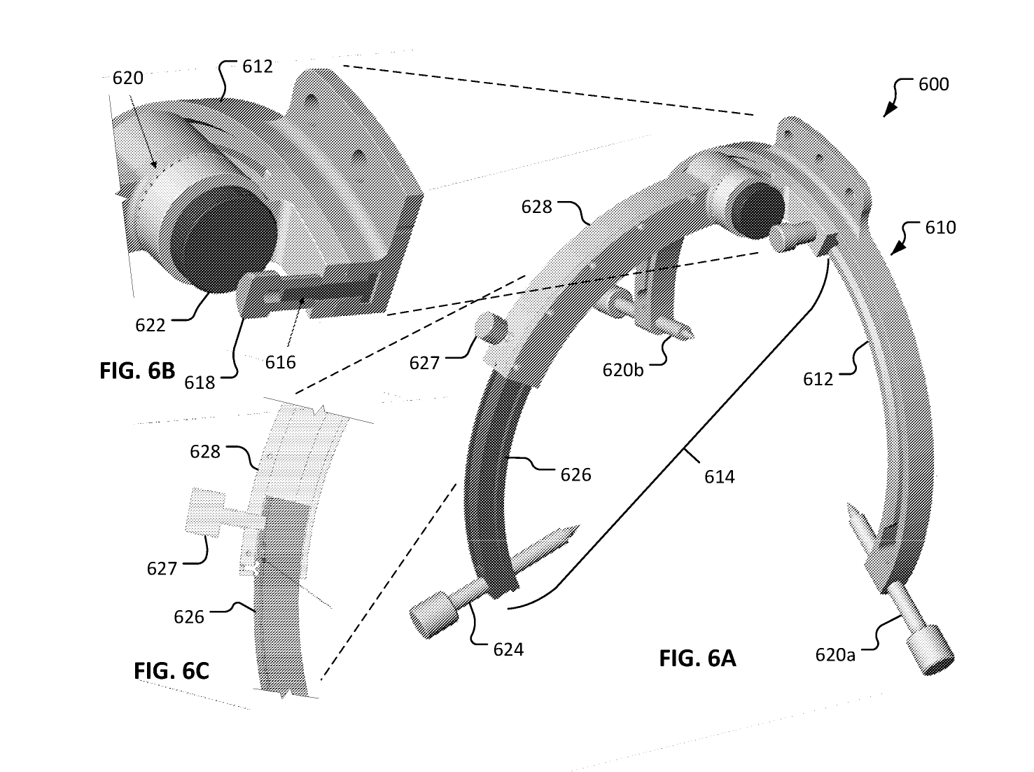

Referring to FIGS. 6A-6C, another example skull traction device 600 provides three-point fixation. In general, skull traction device 600 includes a framework 610 and three pressure-controlled pins: a first temporal pin 620a, a second temporal pin 620b, and an occiput pin 624. First temporal pin 620a and second temporal pin 620b are generally in apposition to each other. Occiput pin 624 can be disposed essentially equidistant between first temporal pin 620a and second temporal pin 620b, but such positioning is adjustable.

Framework 610 includes a U-shaped portion 612 that spans between first temporal pin 620a and second temporal pin 620b. U-shaped portion 612, by itself, is essentially configured as a GW tongs. Added to U-shaped portion 612 is an occipital frame member 614 that is adjustably engageable with the U-shaped portion 612 in this embodiment. For example, occipital frame member 614 can be positioned to extend from U-shaped portion 612 in an orientation so that occipital frame member 614 supports and positions occiput pin 624 to engage with the occipital bone of a patient in the midline. While in the depicted embodiment a single occiput pin 624 is included, in some embodiments two or more occiput pins 624 are included. When multiple occiput pins 624 are included, the multiple pins 624 can be each located on the midline, or the multiple pins 624 can be elsewise orientated generally symmetrically in relation to the midline (e.g., two pins 624 that are laterally positioned on each side of the midline, and so on).

U-shaped portion 612 and first and second temporal pins 620a and 620b can be constructed and configured analogous to U-shaped portion 112 and first and second temporal pins 120a and 120b of skull traction device 100 as described above. In some embodiments, U-shaped portion 612 and first and second temporal pins 620a and 620b are a conventional GW tongs and occipital frame member 614 is selectively engageable with the U-shaped portion 612.

In the depicted embodiment, occipital frame member 614 and U-shaped portion 612 are selectively engageable in relation to each other. That is, in the depicted embodiment occipital frame member 614 and U-shaped portion 612 can be slidably joined together using a joining mechanism such as T-stud 616, and also separated from each other, as desired. For example, T-stud 616 can be threadably coupled with a locking nut 618 that can be tightened to make occipital frame member 614 locked to U-shaped portion 612, and can be loosened to unlock occipital frame member 614 in relation to U-shaped portion 612. With occipital frame member 614 unlocked in relation to U-shaped portion 612, occipital frame member 614 can be slid along U-shaped portion 612 and then locked in a desired arrangement by tightening locking nut 618. Such adjustability can allow the skull traction devices provided herein to advantageously accommodate different skull anatomy.

In the depicted embodiment, occipital frame member 614 is pivotable. By pivoting occipital frame member 614, the result is that occipital frame member 614 is pivoted in relation to U-shaped portion 612. A selectively lockable pivot joint 620 is included to facilitate the ability of occipital frame member 614 to pivot in relation to U-shaped portion 612. A locking screw 622 is used to lock and unlock lockable pivot joint 620. When occipital frame member 614 has been pivoted into a desired relationship with U-shaped portion 612, locking screw 622 can be tightened to lock occipital frame member 614 in that position. Such adjustability can allow the skull traction devices provided herein to advantageously accommodate different skull anatomy.

In the depicted embodiment, occipital frame member 614 is selectively extendable and retractable by sliding a sliding arm 626 in relation to a base arm 628. Occipital frame member 614 includes sliding arm 626 and base arm 628 which are selectively lockable in relation to each other using a locking thumb screw 627. While locking thumb screw 627 is loose, sliding arm 626 and base arm 628 are free to slide in relation to each other, thereby extending or retracting the length of occipital frame member 614. With the length of occipital frame member 614 adjusted to a desired length, locking thumb screw 627 can be tightened to lock sliding arm 626 in relation to base arm 628.

In some embodiments, as an alternative to, or in addition to, thumb screw 627, a mechanism can be included that can be manipulated to extend and retract sliding arm 626 in relation to base arm 628. In some embodiments, such a mechanism is a ratcheting mechanism (such that the location of sliding arm 626 in relation to base arm 628 is incrementally adjustable and will remain fixed after adjustment). In some embodiments, a rack and pinion gear system is included. Including such mechanism(s) will allow sliding arm 626 to be moved relative to the base arm 628 in order to change the flexion/extension rotation of the head after the skull traction device 600 is applied.

Now having described in detail various embodiments of the skull traction devices above, it should be understood that one or more features described in reference to one embodiment can be combined with one or more features described in reference to one or more other embodiments. In result, hybrid designs that combine any or all features described above are envisioned, and such hybrid designs are within the scope of this disclosure.

In some embodiments, some or all components of the skull traction devices provided herein are disposable items (e.g., intended to be single-use components). In some embodiments, some or all components of the skull traction devices provided herein are reusable items (e.g., intended to be multiple-use components). In some such embodiments, the reusable components are sterilizable (e.g., using an autoclave).

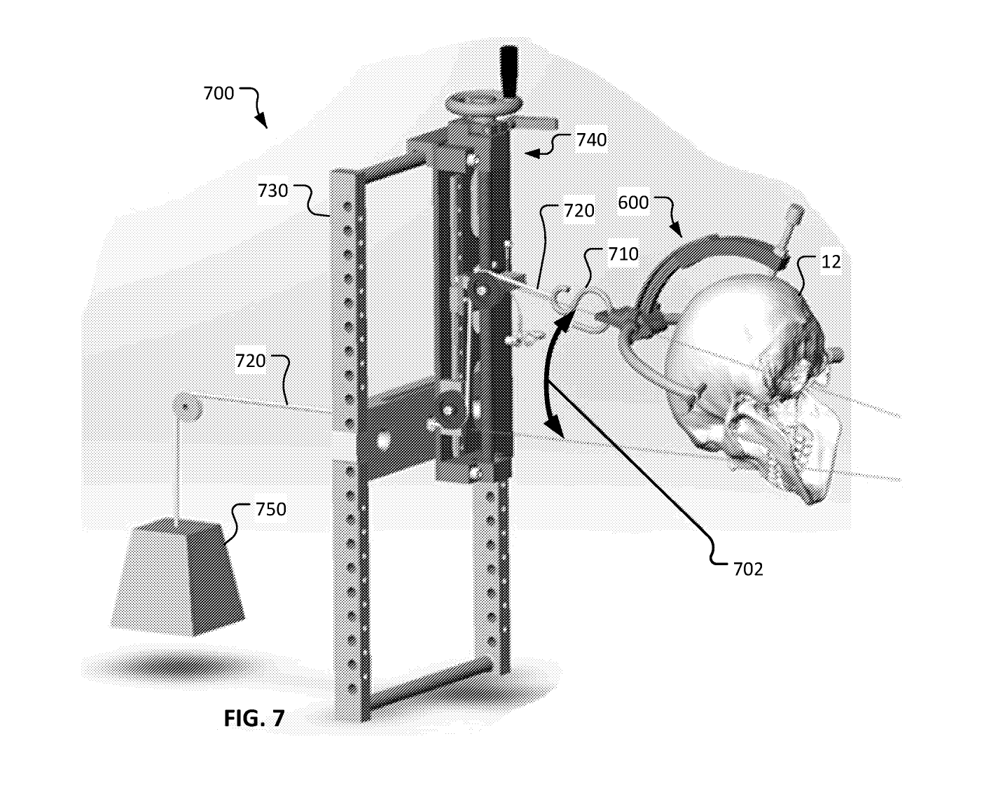

Referring to FIG. 7, an example cervical traction system 700 can be used in conjunction with example skull traction device 600 (or with any of the skull traction device embodiments provided herein) to exert a traction force on skull 12 and, in turn, to the spine of a patient (e.g., refer to FIG. 1). In general, cervical traction system 700 includes a coupler 710, a traction rope 720, an H-frame 730, an adjustable pulley mechanism 740, and a traction weight 750.

In the depicted embodiment, coupler 710 can be used to interconnect skull traction device 600 and traction rope 720. In some embodiments, coupler 710 is not used and traction rope 720 is attached directly to skull traction device 600. Traction weight 750 is attached to traction rope 720. Accordingly, traction weight 750 creates a tensile force in traction rope 720 that is transferred to skull traction device 600 and, in turn, to skull 12 to result in a longitudinal force to the patient's spine. The traction rope 720 is also referred to herein as an elongate traction force application member. In some embodiments, traction rope 720 can be a cable, chain, a rigid rod, and the like.

In some embodiments, H-frame 730 is attached to, or is in a spatially fixed relationship with, an operating table on which the patient is lying (table not shown; refer to FIG. 1 for an example). Adjustable pulley mechanism 740 is coupled to H-frame 730. In the depicted embodiment, adjustable pulley mechanism 740 (as a whole) is fixedly attached to H-frame 730. Alternatively, in some embodiments adjustable pulley mechanism 740 (as a whole) is movably attached to H-frame 730. That is, in some embodiments adjustable pulley mechanism 740 (as a whole) can be selectively positioned (e.g., side-to-side, vertically, angularly, etc.) in relation to H-frame 730 and then locked in a desired relative position.

Adjustable pulley mechanism 740 can be adjusted to cause an adjustment in a traction angle 702. Traction angle 702 is the angle between traction rope 720 and horizontal. For example, in the depicted arrangement, traction angle 702 is about 30°. As described further herein, adjustable pulley mechanism 740 can be selectively adjusted to lower traction angle 702 to about 0° and can be adjusted to increase the traction angle 702 to about 45° (or anywhere therebetween).

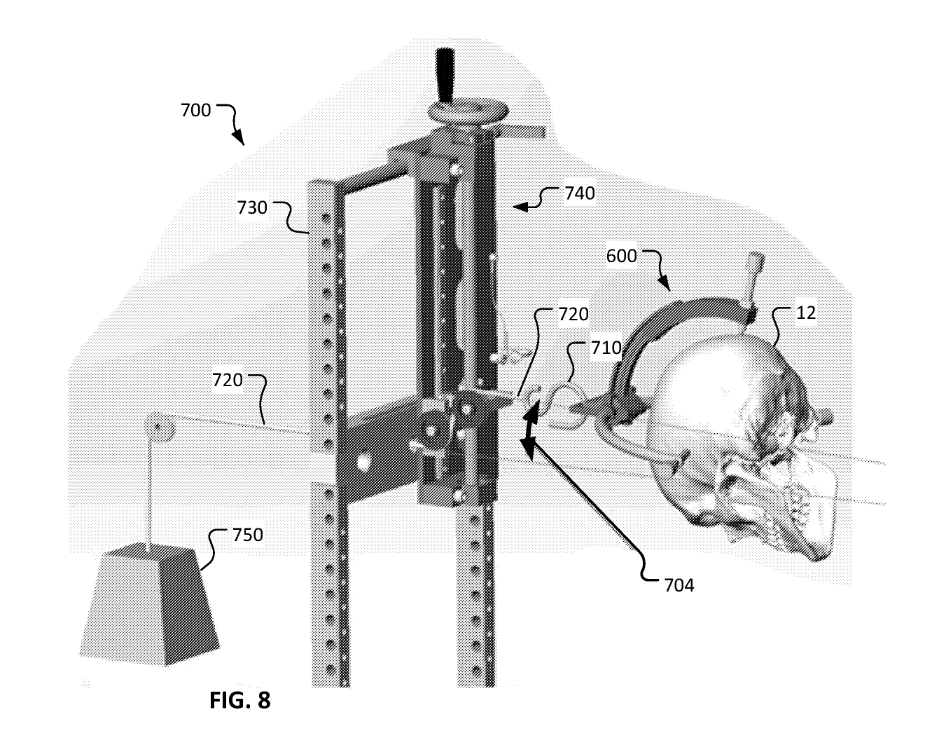

Referring also to FIG. 8, adjustable pulley mechanism 740 can be adjusted to decrease traction angle 702 (as shown in FIG. 7) to lower a traction angle 704 (as shown in FIG. 8). In the arrangement depicted in FIG. 8, traction angle 704 is about 10°. As described further herein, adjustable pulley mechanism 740 can be selectively adjusted between about 0° and about 45° (and anywhere therebetween).

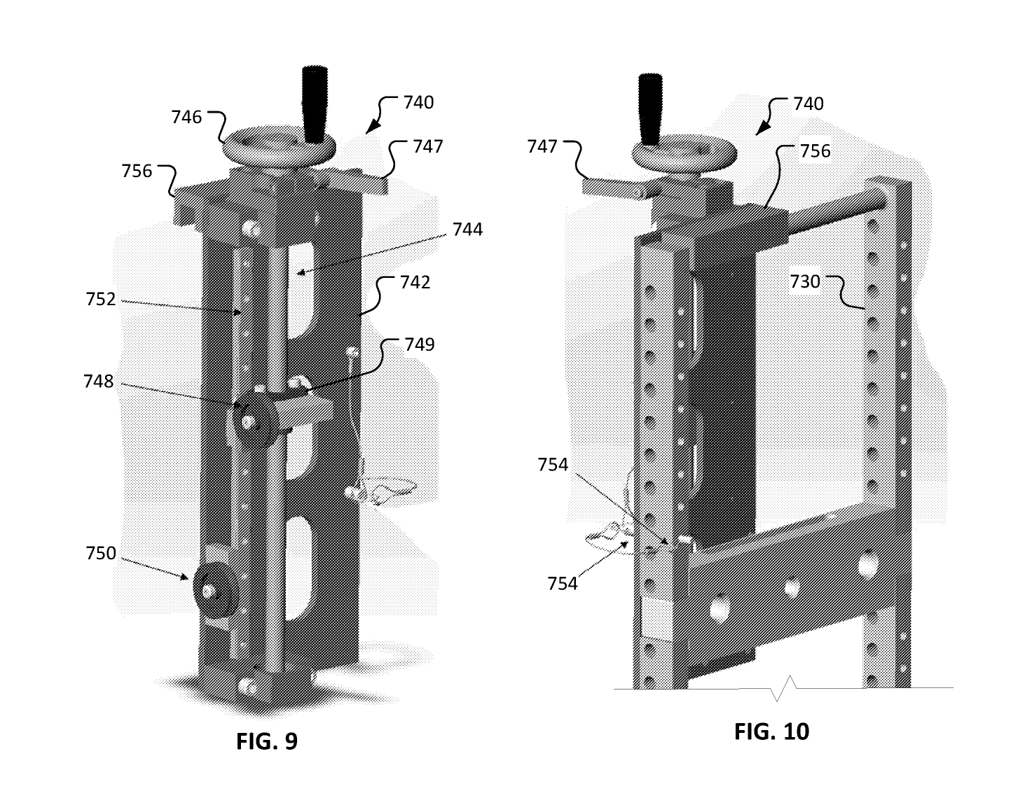

Referring to FIGS. 9 and 10, in the depicted embodiment adjustable pulley mechanism 740 includes a base plate 742, a lead screw 744, a rotary actuator 746, a traction vector pulley 748, a fixed pulley 750, a guide rail 752, a latch 754, and a coupling member 756.

Coupling member 756 performs the function of coupling adjustable pulley mechanism 740 to H-frame 730, and latch 754 interlocks coupling adjustable pulley mechanism 740 to H-frame 730 in a desired arrangement. While in the depicted embodiment, adjustable pulley mechanism 740 is affixed to H-frame 730 in a particular relative arrangement, in some embodiments the relative arrangement of adjustable pulley mechanism 740 to H-frame 730 is adjustable (e.g., side-to-side or up and/or down). Such adjustability can be facilitated manually, motorized, or a combination of both.

In the depicted embodiment of adjustable pulley mechanism 740, fixed pulley 750 is in fixed relationship with base plate 742.

In the depicted embodiment of adjustable pulley mechanism 740, lead screw 744 is threadably engaged with a block 749 to which traction vector pulley 748 is mounted. As lead screw 744 is rotated about its longitudinal axis, block 749 and traction vector pulley 748 translate along lead screw 744. Such translation of traction vector pulley 748 results in an adjustment to the traction angle 702/704 (FIGS. 7 and 8). Guide rail 752 is slidably engaged with block 749 to prevent block 749 from rotating along with lead screw 744. Such rotation of lead screw 744 can be facilitated manually, motorized, or a combination of both. While in the depicted embodiment rotary actuator 746 for rotating lead screw 744 is a hand crank, in some embodiments rotary actuator 746 is a motor or another type of rotary actuator.

In the depicted embodiment, a locking mechanism 747 is included. Locking mechanism 747 can be used to prevent lead screw 744 from being rotated. That is, locking mechanism 747 can lock lead screw 744 (and, in turn, traction vector pulley 748) in a desired location.

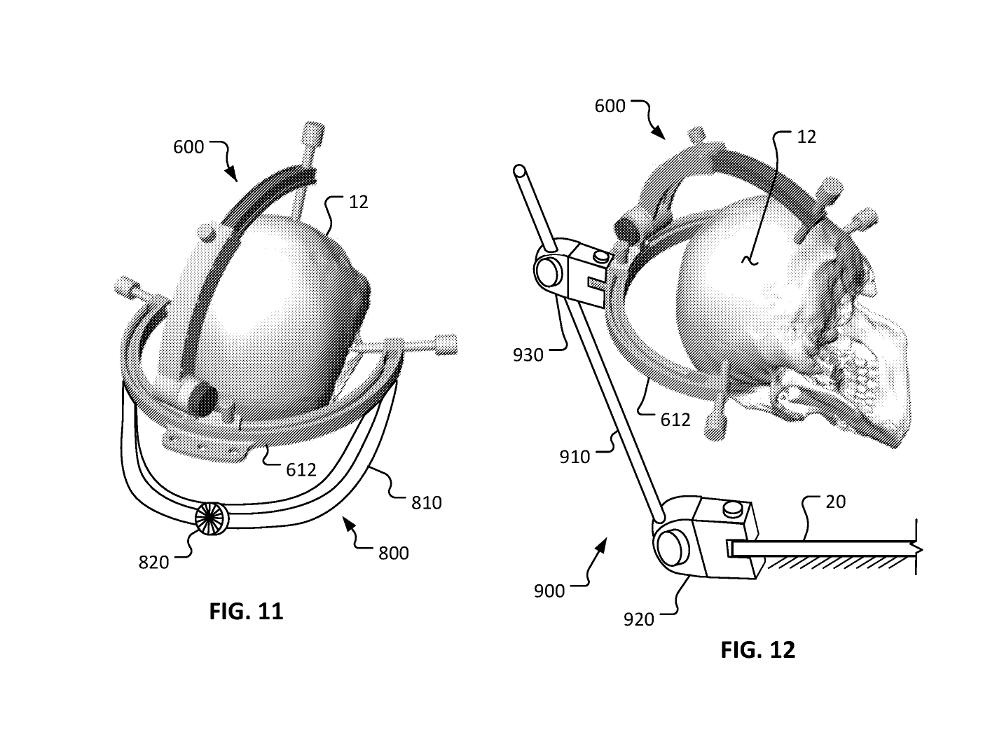

Referring to FIGS. 11 and 12, mechanisms can be included to securely fix the skull apparatus 600 (and others provided herein) in space once the correct position has been achieved. For example, in some implementations the skull apparatus 600 can be adjustably fixed to the operating table on which the patient is lain. The purpose of this enhancement is to lock the patient's skull in a fixed position to prevent loss of alignment during the operation as my occur with muscle relaxation, changes in position of the body over time due to changes in the position of the table (e.g., trendelenberg, reverse trendelenberg, lateral rotation, etc.) or surgical manipulation or destabilization of the spine. Locking the skull position will also improve the accuracy of image guidance.

As shown in FIG. 11, an interface portion 800 can be attached to U-shaped portion 612 of skull traction device 600. Although not required, in the depicted embodiment interface portion 800 extends at least somewhat anterior of skull 12. Using interface portion 800, skull traction device 600 can be releasably, rigidly attached to a stationary object such as a frame or operating table. Accordingly, interface portion 800 can be used to adjustably fix skull 12 in a stationary position.

In the depicted embodiment, interface portion 800 includes an arcuate member 810 and an attachment interface 820. Attachment interface 820 can be fixedly or adjustably attached to arcuate member 810.

While the depicted embodiment includes arcuate member 810 which extends between U-shaped portion 612 and attachment interface 820, it should be understood that other shaped members can be used. For example, instead of the arcuate shape, an L-shape, C-shape, Y-shape, etc., can be used.

In some embodiments, arcuate member 810 is fixedly attached to U-shaped portion 612. In some embodiments, arcuate member 810 is adjustably attached to U-shaped portion 612.

Attachment interface 820 can be used to couple with a fitting of a structural member that is also attached to a rigid object (e.g., operating table, etc.). In some embodiments, attachment interface 820 is a starburst-style locking member that is configured to attach to a complementary starburst-style locking member. In some embodiments, attachment interface 820 can connect to, or in the manner of, a Mayfield device.

As shown in FIG. 12, bracketry 900 can be used to adjustably fix skull traction device 600 (and skull 12) in a stationary position relative to a stationary object such as OR table 20 (or, for example, a frame extending from OR table 20). Bracketry 900 can include one or more articulating arms 910 that secure to the table 20 using standard universal clamps 920 and to the skull apparatus 600 using standard universal clamps 930 at the traction attachment points or other flanges that could be added to U-shaped portion 612.

### Additional Features Included in One or More Embodiments

In some embodiments, one or more aspects of the cervical traction systems provided herein are automated. For example, in some embodiments the design is able to automatically adjust the traction angle to a desired angle that is selected and input by a clinician into a control system. Feedback sensors are included that can measure parameters such as, but not limited to, traction angle. In some embodiments, one or more angular sensors are added to the traction device and/or skull apparatus to detect, for example, the angle of the table relative to the horizontal and angle of the skull apparatus so that preplanned changes in the spine alignment can be made and intraoperative adjustments monitored. These can be independent angular monitors or can be connected with a controller (e.g., programmed with software) to calculate and display their relationship to one another. One or more degrees of freedom of the cervical traction systems can be motorized. The control system of the cervical traction system can receive signals from the feedback sensors and operate the motor and to attain a desired parameter such as traction angle.

While the embodiments described herein move in a single vertical plane to change the position of the head in flexion and extension, in some embodiments the device will attach directly to the skull traction device and manipulate the head and spine through all six degrees of freedom by way of an actuator or multiple actuators. In some such embodiments, a rigid connection member can be used to interconnect the cervical traction system to the skull traction device. Such an arrangement can facilitate, for example, a torque force applied from the cervical traction system to the skull traction device.

In some embodiments, the cervical traction systems provided herein will be motorized and controlled either manually through a hand held remote controller or through a computer software program. Such a software program will allow spinal deformities to be corrected automatically during the course of surgery. Patient specific alignment parameters will be uploaded automatically or manually. Spinal correction will be planned on the software program with various degrees of computerized facilitation. The program will manipulate the spine through the actuators during surgery. The surgeon will be able control the process manually to fine tune the correction or override it for patient safety. The cervical traction systems can include feedback sensors to detect a traction angle and other parameters.

This disclosure also provides devices that will attach to an operating table in order to manually or automatically manipulate the position of the patient's pelvis in relationship to the spine. The purpose of such devices is to facilitate correction of thoraco-lumbar-pelvic deformities. In some embodiments, the device will attach directly to the patient's pelvis through percutaneous pins. In some embodiments, the device will attach indirectly to the patient's pelvis, such as by providing a padded cradle that the patient's hips are placed in. In some embodiments, the patient's pelvis can be manipulated through one, or more degrees of freedom (e.g., two, three, four, five, or six degrees of freedom) by way of an actuator or multiple actuators. Such a device can be used in conjunction with the skull traction devices and cervical traction systems provided herein if so desired, or can be used without the skull traction devices and cervical traction systems provided herein.

The pelvic manipulation devices can be controlled manually through a hand held remote controller or through a computer software program. The software program will allow spinal deformities to be corrected automatically during the course of surgery. Patient specific alignment parameters will be uploaded automatically or manually. Spinal correction will be planned on the software program with various degrees of computerized facilitation. The program will manipulate the spine through the actuators during surgery. For example, in some embodiments the cervical traction system can control both the skull traction devices and the pelvic manipulation devices for comprehensive management of the patient's spinal orientation. The surgeon will be able control the process manually to fine tune the correction or override it for patient safety.

While this specification contains many specific implementation details, these should not be construed as limitations on the scope of any invention or of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described herein as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described herein should not be understood as requiring such separation in all embodiments, and it should be understood that the described program components and systems can generally be integrated together in a single product or packaged into multiple products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In certain implementations, multitasking and parallel processing may be advantageous.

1. A skull traction apparatus comprising:
a framework;
a first temporal pin and a second temporal pin, wherein the first and second temporal pins are engaged with the framework to thereby be positioned and orientated so that the first and second temporal pins can be inserted at generally opposing sides of a patient's skull in the region of the patient's temporal bones; and
an occiput pin engaged with the framework, wherein the occiput pin is engaged with the framework to thereby be positioned and orientated so that the occiput pin can be inserted at the occipital area of the patient's skull when the first and second temporal pins are inserted in the region of the patient's temporal bones,
wherein when each of the first and second temporal pins and the occiput pin are inserted in the patient's skull, the framework is configured for applying a longitudinal force to the patient's spinal column.

2. (canceled)

3. (canceled)

4. The skull traction apparatus of claim 1, wherein the occiput pin is engaged with an occipital frame member portion of the framework, wherein the first and second temporal pins are engaged with another portion of the framework, and wherein the occipital frame member portion is selectively engageable with the other portion of the framework.

5. (canceled)

6. The skull traction apparatus of claim 1, wherein the occiput pin is engaged with an occipital frame member portion of the framework, and wherein the occipital frame member portion is adjustable such that the occiput pin's position is adjustable in relation to the first and second temporal pin locations.

7. The skull traction apparatus of claim 1, wherein the occiput pin is engaged with an occipital frame member portion of the framework, wherein the first and second temporal pins are engaged with another portion of the framework, and wherein the occipital frame member portion is movable in relation to the other portion of the framework.

8. The skull traction apparatus of claim 7, wherein the occipital frame member portion is pivotably movable in relation to the other portion of the framework using a hinge.

9. The skull traction apparatus of claim 7, wherein the occipital frame member portion is slidable along the other portion of the framework.

10. The skull traction apparatus of claim 7, wherein the occipital frame member portion includes a pivot joint that is selectively lockable.

11. The skull traction apparatus of claim 10, wherein the occipital frame member portion includes a first portion and a second portion that are extendable and retractable in relation to each other and that are selectively lockable in relation to each other.

12. A skull traction device comprising:
an occipital frame member, the frame member configured and operable for selective attachment to a U-shaped skull traction device that includes pins for engaging with generally opposing sides of a patient's skull in the region of the patient's temporal bones; and
an occiput pin engaged with the occipital frame member, wherein the occiput pin is engaged with the occipital frame member to thereby be positioned and orientated so that the occiput pin can be engaged with the occipital area of the patient's skull when the occipital frame member is attached to the U-shaped skull traction device and when the pins of the U-shaped skull traction device are engaged with the patient's temporal bones.

13. The skull traction device of claim 12, wherein when the occiput pin and the pins of the U-shaped skull traction device are each engaged with the patient's skull, the combination of the occipital frame member and the U-shaped skull traction device is configured for applying a longitudinal force to the patient's spinal column.

14. The skull traction device of claim 12, wherein the occipital frame member portion is pivotably movable in relation to the U-shaped skull traction device using a hinge.

15-19. (canceled)

20. A cervical traction system configured for applying a longitudinal force to a patient's spinal column, the system comprising:
a skull traction apparatus including one or more pins for engaging with the skull of the patient;
an elongate traction force application member connectable to the skull traction apparatus, wherein, while the traction force application member is in tension and the skull traction apparatus is engaged with the skull of the patient, the longitudinal force is applied to the patient's spinal column; and
an adjustable mechanism engaged with the traction force application member, wherein the adjustable mechanism is adjustable to change a traction angle measured between the traction force application member and horizontal.

21. (canceled)

22. The system of claim 20, wherein the adjustable mechanism is motorized.

23. (canceled)

24. The system of claim 20, wherein the traction force application member is a rope or a cable.

25. (canceled)

26. The system of claim 20, further comprising one or more sensors for detecting the traction angle.

27. The system of claim 26, wherein the adjustable mechanism includes a motor for adjusting the traction angle.

28. The system of claim 27, further comprising a control system for receiving input from the one or more sensors and for sending output to operate the motor in response to the input.

29. The system of claim 28, wherein the control system operates cervical traction system to attain a selected traction angle that is input to the control system.

30. (canceled)

31. The system of claim 20, further comprising a pelvis manipulation apparatus for engaging with a pelvis of the patient such that the pelvis manipulation apparatus can maneuver the pelvis.

32. The system of claim 20, further comprising bracketry attachable to the skull traction apparatus and to a stationary object, wherein the bracketry is configured to releasably fix the skull traction apparatus to the stationary object so that the skull traction apparatus is held stationary.

33. (canceled)
